(19) United States
(12) Patent Application Publication
(10) Pub. No.: US 2019/0242841 A1
(43) Pub. Date: Aug. 8, 2019

(54) HYDROPHOBIC AND OLEOPHOBIC COVER FOR GAS SENSING MODULE

(71) Applicant: IDT INC., San Jose, CA (US)

(72) Inventor: Christian MEYER, Dresden (DE)

(21) Appl. No.: 16/337,382

(22) PCT Filed: Sep. 27, 2017
(86) PCT No.: PCT/EP2017/074508

§ 371 Date: Mar. 27, 2019

(30) Foreign Application Priority Data
Sep. 29, 2016 (DE) 10 2016 118 410.1

(51) Int. Cl. G01N 27/12; G01N 33/00; G01D 11/24

(52) U.S. Cl. CPC G01N 27/125; G01D 11/245; G01N 33/0047

(57) ABSTRACT

The invention discloses a waterproof sensor module. The object of the invention to provide a sensor with a package that is waterproof, in particular absolutely waterproof, but at the same time permeable to target gases that should detected by the sensor will be solved by a sensor comprising a hydrophobic and oleophobic cover that is permeable to gas and waterproof, in particular absolutely waterproof.

### RELATED APPLICATIONS

This patent application is a U.S. National Stage patent application of International Patent Application No. PCT/EP2017/074508, filed on Sep. 27, 2017, which claims priority to German Patent Application No. 10 2016 118 410.1, filed on Sep. 29, 2016, each of which is incorporated by reference herein in its entirety.

### TECHNICAL FIELD

The invention relates to a sensor module that comprises a hydrophobic and oleophobic cover that is permeable to gas and absolutely waterproof.

### DISCUSSION OF RELATED ART

Gas sensors are commonly used for sampling air quality for various gasses. As sensors usually include a sensing element that is exposed to the air to be sampled. The sensing element can provide a signal related to the concentration of the gas detected.

There is a need for better quality detectors.

### SUMMARY

In accordance with certain embodiments, a sensor module that comprises a hydrophobic and oleophobic cover that is permeable to gas and is waterproof is presented. In some embodiments, the cover is a membrane.

These and other embodiments are discussed below with respect to the following figures.

### BRIEF DESCRIPTION OF THE FIGURES

FIG. 1 illustrates a schematic drawing of a gas sensor to detect VOC.

FIG. 2 illustrates potential integration solutions for a waterproof sensor, either a) a waterproof system solution or b) protection of the sensor itself.

FIG. 3 illustrates setup of Gas Permeation Test.

FIG. 4 illustrates measured sensitivity (signal ratio) for different membranes and different gases at different concentrations.

### DETAILED DESCRIPTION

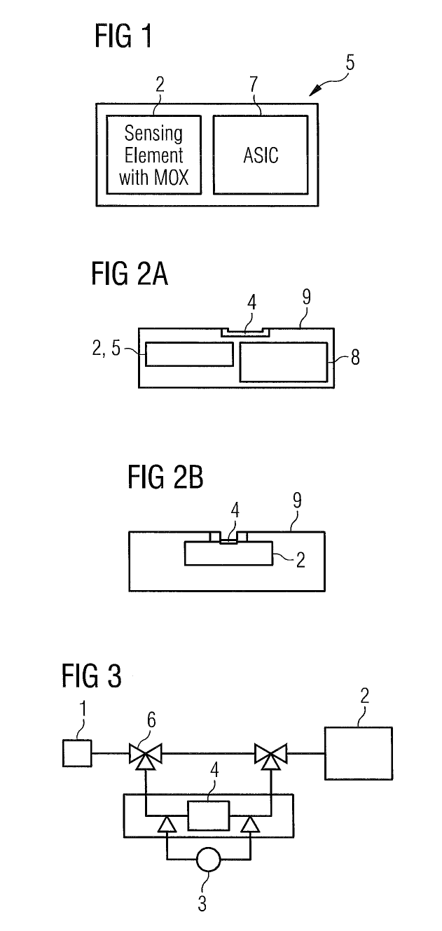

The sensor of the sensor module can be a gas and indoor-air quality sensor that comprises a metal oxide (MOX) gas sensing element and an application specific signal conditioning integrated circuit (ASIC). The sensing element comprises a heater element and a MOX resistive-type sensor supported on a MEMS technology die. The sensor will measure the MOX conductivity, which is a function of the gas concentration. The ASIC has the capability to provide a variety of measurement options; for example, the heater temperature, which may be varied via looped sequencer steps to improve the accuracy of the gas measurements. The MOX sensor temperatures can be selected to optimize sensitivity of different gases: Volatile organic components (VOC), such as Ethanol, Toluene, Formaldehyde, Acetone, and breath Alcohol. The output from the sequencer steps is via I²C™ to the user's microprocessor, which processes the results to determine gas concentration (FIG. 1).

Special Features of the sensor module can be:
Programmable measurement sequence, single shot and automatic cycling of measurements with end-of-sequence interrupt output;
Extremely low current consumption in the μW range;
Heater driver and regulation loop for constant heater voltage or constant heater resistance;
Multiplexed input channel for heater, resistance, and temperature measurements;
Internal auto-compensated temperature sensor, not stress sensitive;
I²C™ interface: up to 400 KHz;
ADC (Analog-to-digital converter) resolution is adjustable for optimal speed versus resolution: 16-bit maximum;
Configurable alarm/interrupt output with static and adaptive levels;
Automatic configuration and measurement start allows fully autonomous operation;
Built-in nonvolatile memory (NVM) for user data;
No external trimming components required, this means that all components of the sensor are trimmed internally and calibrated during a final test in order to be temporally synchronized;
External reset pin (low active);
Detection of VOC with excellent sensitivity to gases like Ethanol, Formaldehyde, Acetone, and Toluene;
Excellent for low-voltage and low-power battery applications;
Customization for mobile and consumer applications.

Some applications require a waterproof system solution to protect electrics against water (IP68—IP protection class 68, meaning dust-tight and resistant to submergence) while detecting different gases, e.g. air quality in very humid environments. Although products are usually waterproofed at the system level, occasionally customers request sensors or sensor modules that are waterproof, requiring a solution to keep out water while allowing gas to enter.

Until now, it is difficult to provide sensors or sensor modules, especially gas sensors which are absolutely waterproof, but are permeable to gases or gaseous media that have long molecules chains.

It is therefore the objective of the invention to provide a sensor or sensor module with a package that is waterproof, in particular absolutely waterproof, but at the same time permeable to target gases that should detected by the sensor.

The objective will be solved by a sensor module comprising a hydrophobic and oleophobic cover that is permeable to gas and waterproof, in particular absolutely waterproof.

In a special embodiment the cover is a membrane. This membrane is waterproof, but molecules with organic chains can pass through, meaning that the membrane is permeable for volatile organic components and molecules with long organic chains.

The membrane can be connected to or stacked to a sensor package, whereas the sensor package comprises a housing and for example a metal surface as a cover. It is also possible to use the membrane itself as a cover for the sensor, e.g. that the membrane itself forms a part of the sensor housing and no separate metal sensor cover is necessary anymore. It is advantageous if the membrane has a thickness of a few μm and has a flow resistance that is 1.0 to 1.25 of the flow resistance without any membrane and the membrane has a high diffusion. A high diffusion means that the diffusion is high enough to avoid a concentration gradient. A thickness of a few μm means 0.2 μm to 0.5 μm. This is necessary to be sensitive against gases that should be measured.

In one embodiment the cover comprises a coating that is hydrophobic and oleophobic. So, it is also possible to attach a coating on a layer that is hydrophobic and oleophobic, meaning it has a reliable protection against water and other corrosive liquids but at the same time the layer is permeable to the target gases.

It is important that the cover tightly closes a surface of the sensor and shields the sensor from a surrounding environment. All substances, e.g. gases can pass the cover but the sensor is not influenced by something else that surrounds the sensor. Such a membrane may be placed on the sensor or the sensor module.

Therefore, the cover is adhered to not active parts of the sensor or to the sensor surrounding by an adhesive or by clamping. Active parts of the sensor are such parts of the sensor that are used for the gas measurement or the ASIC for electronic control; the larger the membranes surface of the sensor the higher the sensor signal. The adhesive can be glue that is chemically inert. It is important that the glue or adhesive is chemically inert and does not outgas, because the waterproof sensor should be long-term stable. It must not react to glue solvents (FIG. 2), because the sensor should detect components in the air.

Several tests have been performed to determine the suitability of two different waterproof membrane samples with different adhesives (acrylic and silicone) and different backing materials. The focus of this investigation was: (1) Test of gas permeation for special gases and (2) Chemical stability of the material for these gases.

All tests have been performed twice with two membranes each. The test gases Acetone, Ethanol and Toluene and the liquids Acetone, Ethanol, Toluene have been applied to the membrane surfaces.

Procedure Gas Permeation Test

The aim of this test was to see the overall ability of the membranes to pass the above mentioned gases. Therefore, a bypass had been intervened to use the maximum membrane surface and not get limited by the smaller pinhole size of the gas sensor. This results in a faster diffusion.

Test gases (Acetone, Ethanol and Toluene) were supplied in high purity in cylinders and diluted via calibrated Mass Flow Controllers with Clean Dry Air. The pipes have been heated to approximately 60° C. to avoid condensation and adsorption. Two 3-way valves give the possibility for a fast switch and test the sensors reaction to gas with and without membrane inside the gas flow. Additionally, a pressure gauge was installed to measure a pressure loss in the gas flow (FIG. 3).

Test Sequence:

Ambient temperature: 25° C.

Sensor operation temperature: 200° C.-450° C.

Flow rate: 0.25 l/min

Relative humidity: 20%

Test time for each gas step: 10 min

Gas steps:
Clean Dry Air
5 ppm Acetone
20 ppm Acetone
Clean Dry Air
5 ppm Ethanol
20 ppm Ethanol
Clean Dry Air
5 ppm Toluene
20 ppm Toluene
Clean Dry Air.

After this gas steps were executed the valves were turned into the bypass position. Exactly the same sequence was started again but now having the membrane with maximum surface inside the gas flow.

For analysis, slope and intercept values were calculated as well as the signal change (ratio R_{Air}/R_{Gas}) for applying the different gas concentrations, whereas R_{Air} is MOX Resistance in Air and R_{Gas} is the MOX Resistance in Gas.

Results Gas Permeation Test:

All tested gases pass the membrane and no limitation of VOC diffusion thru the membrane was observed.

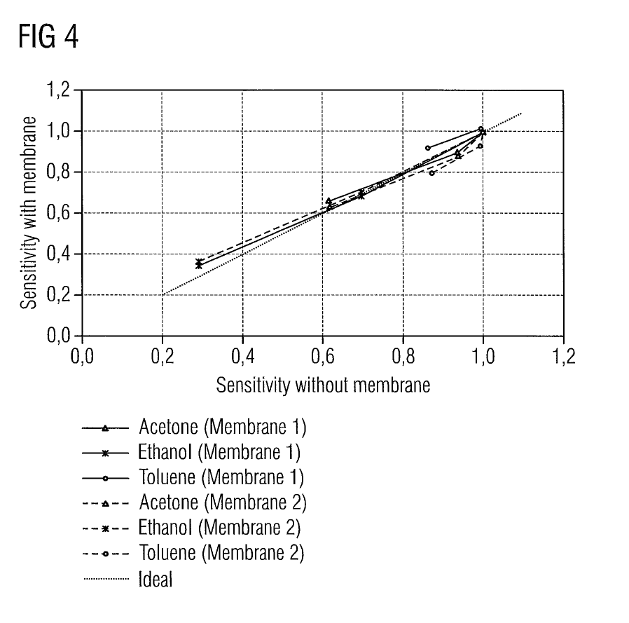

Further analysis proves that sensitivity (signal ratios) slopes and intercept show normal behavior within the limits of accuracy of the sensor operation (FIG. 4).

The pressure difference was detected separately after all gas tests have been finished. It was found that a thicker membrane results in a higher pressure loss. Hence, a gas exchange is more difficult. All membranes used show a low but constant pressure loss; thus no major adsorption or obstruction on the membranes surfaces took place.

Procedure Chemical Stability:

Drops of the liquids Acetone, Ethanol and Toluene (equivalent to target gases) have been placed on top of the membranes to simulate very high concentrations. After 5 min the membrane was visually inspected using a microscope. The inspection was repeated some hours later again.

Results Chemical Stability:

A strong delamination during exposure to Acetone has been observed at the adhesive layer made of silicone. No observation has been made at the membrane using an acrylic adhesive type; the membrane remained intact.

### CONCLUSION

Several tests have been performed to determine the suitability of VOC permeation for two membranes, with different adhesives (acrylic and silicone) and backing material. Gas permeability, pressure loss and chemical stability were investigated and analyzed for the exemplary VOC's Acetone, Ethanol and Toluene.

Both membranes show permeation for all target gases. The small variations in sensor signal with and without membrane are most likely due to sensor performance and are within the sensor accuracy. After an exposure to gas no visual change on the membranes is observed.

For the chemical stability no change has been observed. The exposure to high concentrated test gases over a period of 7 hours with test membranes and additional reference membranes inside the test chamber did not give any indication for instability. However, when exposed to liquids (simulating the very high concentrations) it's seen that the silicon adhesive shows delamination.

The pressure loss of the membrane with thicker backing material is higher which gives a higher flow resistance. This influences the diffusion and will make it more difficult when placing this membrane on top of a small pinhole on top of the sensor because fast gas changes will result in slower sensor signal changes.

The invention will be explained in more detail using exemplary embodiments.

The appended drawings show

FIG. 1 Schematical drawing of a gas sensor to detect VOC;

FIG. 2 Potential integration solutions for a waterproof sensor; either a) a waterproof system solution or b) protection of the sensor itself;

FIG. 3 Setup of Gas Permeation Test;

FIG. 4 Measured sensitivity (signal ratio) for different membranes and different gases at different concentrations.

FIG. 1 shows a schematically drawing of the gas sensor module comprising a metal oxide (MOX) gas sensing element and an application specific signal conditioning integrated circuit (ASIC). The sensor will measure the MOX conductivity, which is a function of the gas concentration. The ASIC has the capability to provide a variety of measurement options; for example, the heater temperature, which may be varied via looped sequencer steps to improve the accuracy or power consumption of the gas measurements.

FIG. 2 shows potential integration solutions for a waterproof sensor. FIG. 2a shows a waterproof system solution, whereas the gas sensor and further electronics are integrated in a sensor housing and whereas the connection between the sensor system and the surroundings is realized over a pinhole. The pinhole is covered by the inventive waterproof cover that is permeable to the detectable gases.

FIG. 2b shows a protection of the sensor itself. The sensor is covered by the permeable cover which is waterproof.

FIG. 3 shows a setup of Gas Permeation Test. The aim of this test was to see the overall ability of the membranes to pass the above gases like Acetone, Ethanol and Toluene. Therefore, a bypass had been intervened to use the maximum membrane surface and not get limited by the smaller pinhole size of the gas sensor. This results in a faster diffusion.

Test gases (Acetone, Ethanol and Toluene) were supplied in high purity in cylinders and diluted via calibrated Mass Flow Controllers with Clean Dry Air. The pipes have been heated to ca. 60° C. to avoid condensation and adsorption. Two 3-way valves give the possibility for a fast switch and test the sensors reaction to gas with and without membrane inside the gas flow. Additionally, a pressure gauge was installed to measure a pressure loss in the gas flow.

FIG. 4 shows the sensitivity of the sensor with and without membrane for the gases Acetone, Ethanol and Toluene. An ideal membrane in which all VOC gases pass the membrane shows no sensitivity differences and would give a straight line in the figure accordingly. However, due to measurement errors small differences for the recording with and without membrane can be seen. This is a normal behavior within the limits of accuracy of the sensor operation.

### REFERENCE SIGNS

1 Mass flow controller
2 Gas sensor
3 pressure gauge
4 filter membrane
5 sensor module
6 3-way valve
7 application specific signal conditioning integrated circuit
8 other electronics
9 sensor system housing

1. Sensor module comprising a hydrophobic and oleophobic cover that is permeable to gas and waterproof.

2. Sensor module according to claim 1, wherein the cover is a membrane.

3. Sensor module according to claim 2, wherein the membrane is permeable for volatile organic components and molecules with long organic chains.

4. Sensor module according to claim 2, wherein the membrane is connected to a sensor package or an integrated sensor system.

5. Sensor module according to claim 2, wherein the membrane has a thickness of a few μm and a flow resistance that is 1.0 to 1.25 of the flow resistance without any membrane and the membrane has a high diffusion.

6. Sensor module according to claim 1, wherein the cover comprises a coating that is hydrophobic and oleophobic.

7. Sensor module according to one of the former claims, wherein the cover tightly closes a surface of a sensor and shields the sensor from a surrounding environment.

8. Sensor module according to claim 7, wherein the cover is adhered to not active parts of the sensor or to the sensor surrounding by an adhesive or by clamping.

9. Sensor module according to claim 7, wherein the adhesive is glue that is chemically inert and does not outgas.

10. Sensor module according to one of the former claims, wherein the impermeability to water is guaranteed all the time by the membrane and the cover.
